(19) United States
(12) Patent Application Publication
(10) Pub. No.: US 2026/0158091 A1
(43) Pub. Date: Jun. 11, 2026

(54) SYSTEMS AND TECHNIQUES FOR EXTRACTING HEMP SEED OIL

(71) Applicant: Crown Iron Works Company, Blaine, MN (US)

(72) Inventors: Nicholas Ollila, Fridley, MN (US); Wade Steven Martinson, Minneapolis, MN (US); Jeff Garritsen, Forest Lake, MN (US); Austin Nelson, Maple Grove, MN (US); Benjamin Wayne Floan, Andover, MN (US)

(21) Appl. No.: 18/970,601

(22) Filed: Dec. 5, 2024

(51) Int. Cl. A61K 36/185; B01D 11/02; B01D 17/02; B30B 3/04

(52) U.S. Cl. CPC A61K 36/3482; B01D 11/028; B01D 11/0288; B01D 17/0214; B30B 3/04; A61K 2236/15; A61K 2236/17; A61K 2236/333; A61K 2236/37; A61K 2236/51; A61K 2236/53; A61K 2236/55

(57) ABSTRACT

Systems and techniques are provided for processing a high oil concentration feedstock, particularly a hemp feedstock, to extract oil from the feedstock. In some examples, the feedstock is processed by flaking the hemp feedstock followed by solvent extraction. The hemp material may be introduced between rotating flaking rolls to flake the hemp heart feedstock. According to some examples, the speed and/or spacing of the flaking rolls may be controlled to control the characteristics of the resulting flaked hemp material for subsequent solvent extraction. Extraction performance of the hemp feedstock can vary based on the characteristics of the flake properties dictated by controlling the flaking rolls.

### TECHNICAL FIELD

This disclosure relates to solvent extraction of high oil concentration feedstocks and, more particularly, to solvent extraction of hemp material.

### BACKGROUND

A variety of different industries use extractors to extract and recover liquid substances entrained within solids. For example, producers of oil from renewable organic sources use extractors to extract oil from oleaginous matter. The oleaginous matter is contacted with an organic solvent within the extractor, causing the oil to be extracted from a surrounding cellular structure into the organic solvent.

There is increasing awareness of the medicinal, wellness, and nutritional benefits of hemp seed products and extracts. For example, hemp seed oil is a rich source of unsaturated fatty acids, including omega-3 and omega-6 fatty acids. These fatty acids are essential for human health and can help to improve cardiovascular health, reduce inflammation, and promote skin health. Hemp seed oil can be extracted from hemp seeds using solvent extraction.

### SUMMARY

In general, this disclosure is directed to devices, systems, and techniques for processing a high oil concentration feedstock, particularly a hemp feedstock, to extract oil from the feedstock and/or to produce a protein product separated from the extracted oil. In practice, it has been identified that processing high oil concentration feedstocks such as hemp can be challenging for traditional extractive systems and techniques. The high oil concentration of the feedstock can interfere with extraction kinetics, residence times for adequate oil recovery and subsequent solvent recovery, and the processability of the feedstock.

In accordance with examples of the present disclosure, systems and techniques are described for extracting high oil concentration feedstocks. The example systems and techniques are described with particular reference to a hemp feedstock although can have applicability for other feedstocks, such as other feedstocks having a comparatively high oil concentration. The disclosed systems and techniques can provide increased yield and extraction efficiency compared to alternative solvent extraction techniques, resulting in enhanced recovery, feedstock utilization, and process economics for the operator and resulting consumer.

In some examples, the disclosed systems and techniques provide a continuous process for producing oil from hemp seeds. The hemp seeds can be processed to dehull the hemp seeds and to separate hemp hearts from the hemp seeds. The hemp hearts can then be processed for solvent extraction in a continuous solvent extractor. The hemp hearts can be flaked to size reduce the hemp hearts to produce a flaked hemp material that is subsequently introduced into the solvent extractor. The hemp hearts can be introduced between rotating flaking rolls to flake the hemp heart feedstock. According to some examples of the present disclosure, the speed and/or spacing of the flaking rolls may be controlled to control the characteristics of the resulting flaked hemp material for subsequent solvent extraction. In practice, it has been observed that the extraction performance of the hemp feedstock can vary based on the characteristics of the flake properties dictated by controlling the flaking rolls.

For example, the thickness of the hemp flakes may be controlled within a set range. In general, reducing the thickness of the hemp flakes increases surface area for fast extraction kinetics. However, the smaller surface area flakes can retain solvent for longer than comparatively larger flakes, increasing solvent drainage times following extraction. Moreover, it has been observed that mean flake thickness can be unexpectedly larger than the spacing set between adjacent flaking rolls, guiding toward the use of smaller spaced flaking rolls to achieve a particular target flake size.

In addition to or in lieu of controlling the spacing between the flaking rolls, the speed of the flaking rolls may also be controlled to control the extraction characteristics of the resulting flaked material. In some implementations, it has been observed that controlling the speed of the flaking rolls can impact the oil extraction characteristics of the flaked material. Flaking the hemp material at a comparatively slower flaking roll speed can cause more oil to be extracted from the resulting flaked material compared to hemp material flaked using faster roll speeds under otherwise similar conditions extraction conditions.

A variety of additional and/or different extraction systems and techniques may be implemented according to the disclosure to enhance the efficacy of oil extraction from a hemp material. As one example, the hemp material may be processed for subsequent solvent extraction by flaking the hemp material without preconditioning the material prior to flaking. For example, the hemp may be cold flaked (e.g., flaked under ambient conditions) without pre-heating the hemp prior to flaking. Cold flaking the hemp may be beneficial to help prevent agglomeration of the otherwise comparatively hot, high oil content material during and/or after flaking prior to solvent extraction. Agglomeration of the hemp that may be observed when preconditioning the material prior to flaking in certain circumstances can reduce the performance of the flaking operation and the resulting amount of oil extracted from the material during subsequent solvent extraction.

In one example, a method is described that includes flaking a hemp material by passing the hemp material through a pair of rotating flaking rolls defining a nip clearance between the rotating flaking rolls within a range from 0.2 mm to 0.32 mm, thereby producing a flaked hemp material having a mean thickness within a range from 0.25 to 0.65 mm. The method further involves introducing the flaked hemp material into an extractor and contacting the flaked hemp material with a solvent, thereby producing an extracted hemp material and a miscella. The method also involves separating the solvent from the miscella, thereby producing an extracted hemp oil.

In another example, a method is described that includes flaking a hemp material by passing the hemp material through a pair of rotating flaking rolls one or both of which is operating at a speed less than 2 m/s to produce a flaked hemp material. The method further involves introducing the flaked hemp material into an extractor and contacting the flaked hemp material with a solvent, thereby producing an extracted hemp material and a miscella. The method also involves separating the solvent from the miscella, thereby producing an extracted hemp oil.

In another example, a method is described that includes flaking a hemp material at ambient temperature or at a temperature below ambient temperature to produce a flaked hemp material. The method further involves introducing the flaked hemp material into an extractor and contacting the flaked hemp material with a solvent, thereby producing an extracted hemp material and a miscella. The method also involves separating the solvent from the miscella, thereby producing an extracted hemp oil.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a flow diagram illustrating an example process for extracting oil from an oleaginous feed stock.

FIG. 2 is an illustration of an example flaking machine that can be used to flake a feedstock, such as a hemp heart feedstock, according to the disclosure.

FIG. 3 is a side sectional view of an example configuration of the flaking machine of FIG. 2.

FIG. 4 is a block diagram illustrating an example extraction system in which a feedstock material, particularly a hemp seed material, is extracted.

FIG. 5 is an illustration of an example immersion extractor configuration that can be used according to the disclosure.

FIGS. 6A and 6B are plots of cumulative flake thickness distributions and flake thickness distributions for experiments conducted.

FIG. 7A is a plot of cumulative nonvolatiles extracted from flaked hemp samples of different thickness versus time.

FIG. 7B is a plot of cumulative nonvolatiles extracted from flaked hemp samples of different thickness versus cumulative miscella recovered.

FIG. 8 is a plot of solvent concentration in extracted flaked hemp samples versus time for hemp samples of different thicknesses.

### DETAILED DESCRIPTION

In general, the disclosure relates to liquid-solid extractor systems and processes that enable the extraction of one or more desire products from solid material flows. In some examples, the solid material is processed in a continuous flow extractor that conveys a continuous flow of material from its inlet to its outlet while a solvent is conveyed in a countercurrent direction from a solvent inlet to a solvent outlet. As the solvent is conveyed from its inlet to its outlet, the concentration of extracted liquid relative to solvent increases from a relatively small extract-to-solvent ratio to a comparatively large extract-to-solvent ratio. Similarly, as the solid material is conveyed in the opposing direction, the concentration of extract in the solid feedstock decreases from a comparatively high concentration at the inlet to a comparatively low concentration at the outlet.

The solvent discharged from the extractor, which may be referred to as a miscella, contains extracted components (e.g., oil) from the solid feedstock. The solvent-wet solid material discharged from the extractor may be residual solid feedstock having undergone extraction that carries entrained solvent and is referred to as a marc.

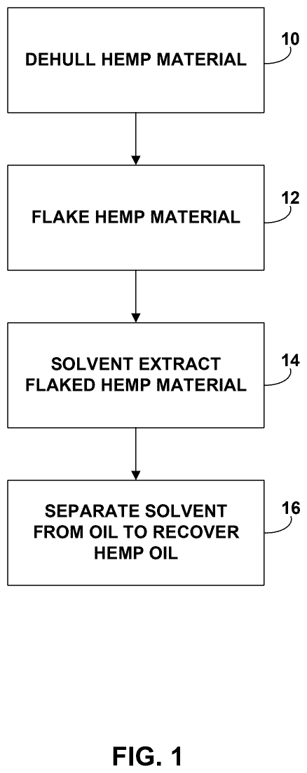

FIG. 1 is a flow diagram illustrating an example process for extracting oil from an oleaginous feed stock. The technique of FIG. 1 will be described specifically with reference to extracting hemp oil from hemp seeds, although can be performed to extract different oils from different feedstocks. The example technique includes dehulling hemp seeds to obtain separated hemp hearts (10) and flaking the separated hemp hearts to produce a flaked hemp material (12). The example technique of FIG. 1 further involves introducing the flaked hemp material into an extractor and contacting the flaked hemp material with a solvent to produce an extracted hemp material and a miscella (14). In some examples, the technique further includes separating the solvent from the miscella to produce an extracted hemp oil (16).

To extract oil from a feedstock material, the feedstock material may be preprocessed prior to performing solvent extraction on the feedstock material. Hemp seed oil can be obtained from the seeds of the Cannabis sativa plant. The oil can contain omega-6 and omega-3 fatty acids, alpha-linolenic acid, gamma-linolenic acid, and/or other nutritional antioxidants. Hemp seed oil typically no tetrahydrocannabinol (THC) and little to no CBD. Accordingly, implementations of the process of FIG. 1 may be performed using a hemp seed feedstock to produce a hemp seed oil.

In other implementations, other feedstocks may be used in the example technique of FIG. 1. For example, the feedstock may be a plant material that includes flowering plants in the family Cannabaceae, Cannabis sativa, Cannabis indica, Cannabis ruderalis, hemp or any combination thereof. Typically the flower of the female plant contain the highest quantities of cannabinoids. In still other examples, other non-cannabinoid feedstocks may be used in process of FIG. 1. Example, solid material feedstocks that can be processed in the example technique of FIG. 1 include oleaginous materials, such as soybeans, rapeseed, sunflower seed, peanuts, cottonseed, palm kernels, and corn germ, and other oil-bearing seeds and fruits.

In the example of FIG. 1, the example technique includes dehulling the feedstock material (10). For example, when using a hemp seed feedstock, the hemp seeds can be dehulled to obtain separated hemp hearts. Examples of dehulling methods can include dehulling and cleaning by an impact, roll or raspelling de-huller, aspirator, clipper cleaner, fluidized bed gravity table, sieve, indent cleaning, scalper, and/or the like. In some examples, the dehulling and cleaning step can produce dehulled hemp seeds that are substantially free of hemp seed shells, hemp seed coat and foreign material. Foreign material can be defined as any non-hemp material present on the hemp seeds. Substantially free can be defined as a reduction of at least 50%, 60%, 70%, 80%, 90%, 99%, or more of the hemp seed shells, seed coat and/or foreign material that was present on the hemp seeds prior to the dehulling step.

To obtain separated hemp hearts from the residual foreign material, the hemp hearts can be sorted from the dehulled hemp seeds. The sorting step can include sieving, indent cleaning, scalping, aspiration, clipper cleaners, gravity tables, and/or optical sorting. In some examples, dehulled hemp seeds may be conveyed into a hopper that feeds an optical sorter. The optical sorter can use air flow to remove non-heart material from the dehulled hemp seeds. Non-heart material can include shells, seed coat, fiber, foreign material, and/or the like. The isolated hemp seed hearts after the sorting step can be referred to as white hemp hearts (WHH) or primarily endosperm material or isolated hemp hearts. These hearts are substantially free from all non-heart material including but not limited to the hulls, fractional hull pieces, seed coat (the green covering interior to the hull, exterior to the heart) that cover the hemp endosperm (heart) and foreign materials (rocks, insects, stems, non-hemp seeds).

Hemp hearts provide an oleaginous feedstock material characterized by a high oil content and low carbohydrate content compared to other oleaginous feedstock materials, such as soy. For example, the hemp heart feedstock may contain from 40 weight percent to 60 weight percent oil, such as from 45 weight percent to 55 weight percent oil, or from 47 weight percent to 51 weight percent oil. The hemp heart feedstock may contain less than 12 weight percent carbohydrates, such as less than 10 weight percent carbohydrates, or less than 8 weight percent carbohydrates. For example, the hemp heart feedstock may contain from four weight percent to 10 weight percent carbohydrates, such as from six weight percent to eight weight percent carbohydrates. In some implementations, the hemp heart feedstock contains from 25 weight percent to 40 weight percent protein, such as from 27 weight percent to 37 weight percent protein, or from 30 weight percent to 34 weight percent protein.

The example technique of FIG. 1 includes flaking the hemp heart feedstock to produce a flaked hemp material (12). Flaking the hemp heart feedstock reduces the size of the material and increases the surface area of the hemp heart feedstock for subsequent solvent extraction in a solvent extractor. Flaking reduces the sides of the particles to increase the efficiency of the subsequent solvent extraction process. Flaking can flatten the hemp heart feedstock into thin flakes having a reduced size compared to the size of the incoming feedstock. This reduces the distance that solvent and oil must diffuse in and out of the solid material during extraction. Flaking also ensures more uniform processing of the feedstock by making sure that all of the feedstock particles are substantially uniformly thick.

Flaking of the hemp hear feedstock material may be accomplished by passing the hemp material through a pair of flaking rolls. Different types of flaking roll arrangements may be used. In one example, two flaking rolls are arranged in parallel and driven in rotation in opposite directions so that the two rolls pull the feedstock material down between. Mechanical jackscrews or hydraulic cylinders can apply pressure against bearing blocks supporting the rolls so that the rolls do not spread apart as the feedstock material passes between the rolls. In different implementations, the two rolls may be driven at the same speed, or one roll they be driven at a faster speed than the other roll (e.g., to help prevent the feedstock material from smearing).

In another example implementation, multiple flaking rolls are stacked vertically on top of each other. The vertical stacked arrangement of flaking rolls includes at least two sets rolls and may include more sets of rolls, such as from four to six rolls. The weight of the upper rolls can provide pressure to the lower rolls. In operation, the feedstock material is progressively flattened into thinner and thinner flakes as it passes from roll to roll in descent down the stack.

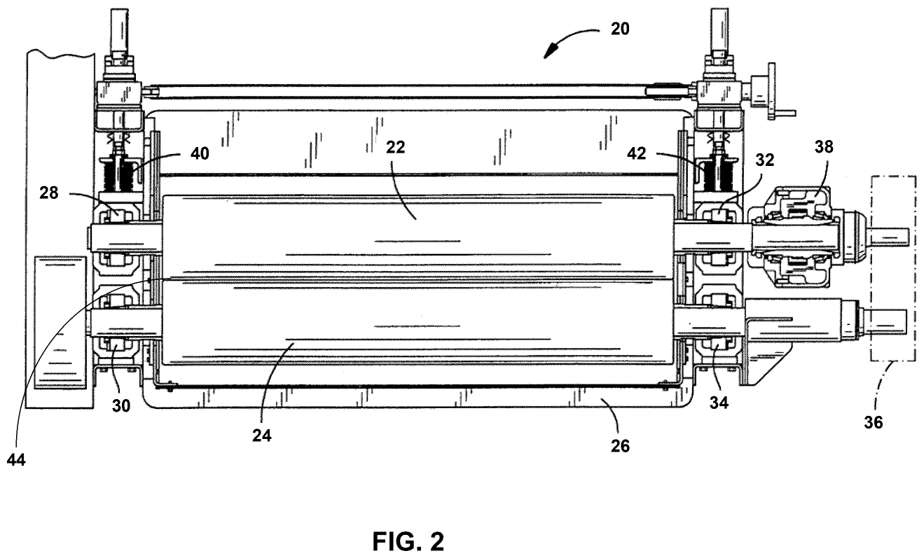

FIG. 2 is an illustration of an example flaking machine 20 that can be used to flake a feedstock, such as a hemp heart feedstock, according to the disclosure. In particular, FIG. 2 illustrates a portion of a flaking machine 20 (which may also be referred to as a milling machine) that includes a pair of co-acting rolls 22 and 24. Each roll 22, 24 may be is journalled in a machine frame 6, and the shafts thereof mounted in bearings 28, 30, 32 and 34. An inter-roll drive 36, which is represented simply by dashed-line outlining, can be a common belt-type drive, that drives the pair of rolls 22, 24. A single, gearbox, inter-roll drive 38 may be mounted to a first roll 22 and operative to drive both rolls 22, 24. Additionally, first roll 22 may be biasingly held against second roll 24 by a biasing mechanism, such as compression springs 40 and 42. During operation, inter-roll drive 36 can maintain a constant force between the rolls 22 and 24 at the right-hand end of the rolls. The spacing 44 between rolls 22, 24, which is also referred to as the nip clearance, defines the distance separating first roll 22 from the second roll 24. The spacing 44 may be adjustable to control the thickness of the flake produced by the flaking machine 20.

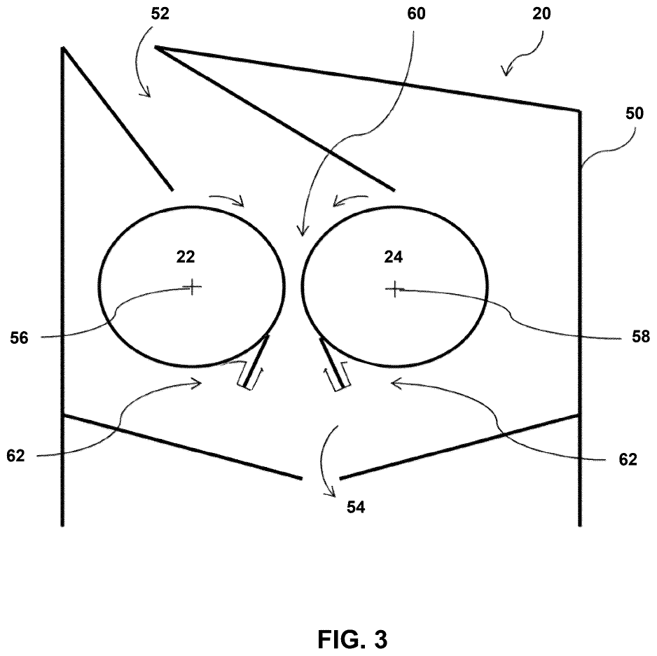

FIG. 3 is a side sectional view of an example configuration of flaking mill 20. As shown, flaking mill 20 can include a housing 50 having a feed inlet 52 and a feed outlet 54. Rolls 22, 24 can be arranged in housing 50 and mounted so as to be rotatable about roll axis 56 and 58, respectively. Downstream of a milling gap 60 defining the nip clearance between the opposed rolls, is one or more scrapers 62. Each scraper 62 may comprise a blade for scraping milling material from a roll surface. The blade can have a scraper edge which extends over the entire length of the blade. The length of the blade may substantially corresponds to the axial length of the roll such that a blade can be used to scrape the entire roll surface. Alternatively, a segmented blade can be used that defines a plurality of elements which, when lined up, can scrape the entire roll surface. In some examples, scraper 62 may be formed of a non-stick material (e.g., a polymeric material) or be fabricated of metal and include a non-stick coating, in each case to reduce sticking and agglomeration between the scraper and flaked feedstock material.

In operation, feedstock material is introduced via feed inlet 52 of housing 50. The feedstock material enters milling gap 60 and is sized reduced down to a flake having a thickness corresponding to nip clearance 44 between rolls 22 and 24. In accordance with some examples of the present disclosure, nip clearance 44 and/or the speed of one or both of rolls 22, 24 may be controlled to control the extraction characteristics of the resulting flaked material.

For instance, in some examples, the feedstock may be flaked down to a flaked feedstock having flakes exhibiting a mean thickness less than 1.0 mm, such as less than 0.75 mm, less than 0.65 mm, less than 0.5 mm, less than 0.45 mm, less than 0.4 mm, less than 0.35 mm, or less than 0.3 mm. While reducing the thickness of the flakes can increase the amount of surface area for contacting the solvent, if the material is flaked to be excessively thin, the material may hold excessive amounts of solvent after extraction causing large solid drainage times and/or excessive solvent entrapment. In some examples, the feedstock may be flaked down to a flaked feedstock having flakes exhibiting a mean thickness greater than 0.15 mm, such as greater than 0.2 mm, greater than 0.25 mm, greater than 0.3 mm, greater than 0.35 mm, greater than 0.4 mm, greater than 0.45 mm, or greater than 0.5 mm.

For instance, in some examples, the feedstock is flaked down to a flaked feedstock having flakes exhibiting a mean thickness within a range from 0.2 mm to 0.75 mm, such as from 0.25 mm to 0.65 mm, 0.30 mm to 0.65 mm, 0.32 mm to 0.6 mm, or 0.35 mm to 0.55 mm. In some implementations, the distribution of flake thicknesses is comparatively narrow to provide good uniformity across the entire flaked feedstock. For example, the flaked feedstock may exhibit a standard deviation less than 0.12 mm, such as less than 0.11 mm, less than 0.10 mm, less than 0.09 mm, or less than 0.08 mm.

The thickness of the flakes produced by the flaking machine (e.g., flaking machine 20) can be controlled by controlling nip clearance 44 between rolls 22 and 24. In practice, it has been observed that some feedstocks, such as hemp hearts, exhibit a mean flaked thickness larger than the nip clearance 44 set between opposing rolls 22 and 24. In other words, the feedstock material does not flake down to a thickness that is the same as the clearance or distance between opposing rolls. Rather, the flaked feedstock may exhibit a larger thickness than the nip clearance 44 set between opposing rolls. Without wishing to be bound by any particular theory, it is believed that the feedstock may elastically compress during flaking, allowing the flakes produced by flaking machine 20 to expand after flaking to a thickness greater than that set by nip clearance 44.

Accordingly, in some implementations, the nip clearance 44 between opposed rolls 22 and 24 may be set smaller than the target mean flake thickness desired to be achieved through flaking. In some examples, the mean flake thickness achieved through flaking may be within a range from 1.5 times to 3.0 times larger than nip clearance 44, such as within a range from 1.5 to 2.5 times larger than nip clearance 44. Accordingly, nip clearance 44 on flaking machine 20 may be set within a range of 0.2 to 0.8 times the mean flake thickness desired to be achieved through flaking (which includes any of the flake thicknesses and ranges discussed above), such as from 0.3 to 0.7 times the mean flake thickness, or from 0.3 to 0.5 times the mean flake thickness. In some examples, the clearance 44 is within a range from 0.12 mm to 0.4 mm, such as from 0.2 mm to 0.35 mm, or from 0.2 mm to 0.32 mm.

Independent of the nip clearance 44 set for flaking machine 20 and the corresponding mean flake thickness produced by the flaking machine, the speed of first roll 22 and/or second roll 24 may be controlled to help enhance the extraction characteristics of the resulting flaked material. In some examples in practice, it has been observed that driving first roll 22 and/or second roll 24 at a comparatively slower speed results in the flaked material containing less residual oil following extraction than when flaking at comparatively higher speeds under otherwise equivalent flake thickness and extraction conditions.

Accordingly, in some implementations, first roll 22 and/or second roll 24 of flaking machine 20 (and, optionally, other rolls when flaking machine 20 includes more than two rolls), may be driven at a comparatively slow speed. For example, first roll 22 and/or second roll 24 of flaking machine 20 may be driven at a speed less than 3 m/s, such as less than 2.5 m/s, less than 2.0 m/s, less than 1.5 m/s, or less than 1.0 m/s. First roll 22 may be driven at the same speed as second roll 24, or first roll 22 may be driven at a different speed than second roll 24.

Independent of the specific configuration and operating parameters of the flaking step, the feedstock material may or may not be conditioned prior to being flaked. During traditional conditioning, the feedstock material is heated prior to flaking and/or extraction. Heating the feedstock material can reduce oil viscosity and rupture oil bodies, facilitating oil extraction. In accordance with some implementations of the present disclosure, however, the feedstock material may be flaked without conditioning the feedstock prior to flaking. This may be referred to as cold flaking.

For example, the feedstock may be flaked at ambient conditions (e.g., ambient temperature and/or pressure) without preconditioning the hemp material, e.g., such that the material being flaked is at ambient temperature and/or pressure. In some examples, the hemp material may even be cooled to a temperature below ambient temperature prior to flaking, e.g., such that the material being flaked is at a temperature below ambient temperature. In various examples, material being flaked may be a temperature of 30° C. or less, such as 25° C. or less, 20° C. or less, 15° C. or less, 10° C. or less, 5° C. or less, or 0° C. or less. Reference to flaking the hemp without preconditioning the hemp can mean that the hemp is not exposed to an elevated temperature in one or more a heater, oven, roaster, conditioner, and/or the like.

Flaking the hemp material at or below ambient temperature can be beneficial for a variety of reasons. If preconditioning the hemp material and hot flaking the material, the hemp may have a tendency to agglomerate, e.g., because of the high oil concentration of the material. This can cause build-up on the flaking machine, leading to operational problems, and inconsistent flaking and a lack of uniformity in the size of the resulting flakes. By contrast, cold flaking the hemp material at or below ambient temperature can reduce material build up on the flaking machine and provide a resulting flake product having a more consistent size distribution for effective solvent extraction.

The flaked hemp material may or may not be dried after flaking prior to being introduced into the extractor for solvent extraction. In some examples, a dryer dries the flaked material before subsequently delivering a dried flaked material to the extractor. For example, a direct or indirect dryer may be used to vaporize moisture from the flaked material prior to delivery to the extractor. When used, the dryer may indirectly dry the flaked solid material, e.g., by passing a thermal transfer fluid through a jacketed drying vessel. Additionally or alternatively, the dryer may directly dry the flaked solid material, e.g., by introducing a hot gas (e.g., dried air, nitrogen) into the dehydrated solid material to vaporize water that is then venting as a gas out of the vessel. When used, a dryer may dry the flaked solid material at a temperature greater than 30° C., such as greater than 50° C., or greater than 60° C., greater than 70° C., greater than 80° C., or greater than 100° C. Additionally alternatively, the dryer may dry the flaked solid material at a temperature less than 125° C., such as less than 100° C., or less than 80° C.

In some examples, the flaked material has a moisture content greater than five weight percent prior to being dried, such as greater than six weight percent, greater than seven weight percent, greater than eight weight percent, greater than nine weight percent, or greater than 10 weight percent. For example, the flaked material have a moisture content ranging from five weight percent to 12 weight percent. A dryer may reduce the moisture content of the flaked solid material by at least 0.5 weight percent, such as by at least one weight percent, by at least two weight percent, by at least three weight percent, by at least four weight percent, or by at least five weight percent. Accordingly, the flaked solid material may have a moisture content less than 5 wt % after drying and prior to being introduced into the extractor, such as less the 4 wt %, less than 3 wt %, or less than 2 wt %.

With further reference to FIG. 1, the example technique also includes introducing the flaked hemp material into an extractor and contacting the flaked hemp material with a solvent to produce an extracted hemp material and a miscella (14). In some examples, the technique further includes separating the solvent from the miscella to produce an extracted hemp oil (16). A variety of different batch and/or continuous extractor systems may be used to solvent extract hemp oil from the flaked hemp material and to recover the extracted hemp oil from the solvent used in extraction.

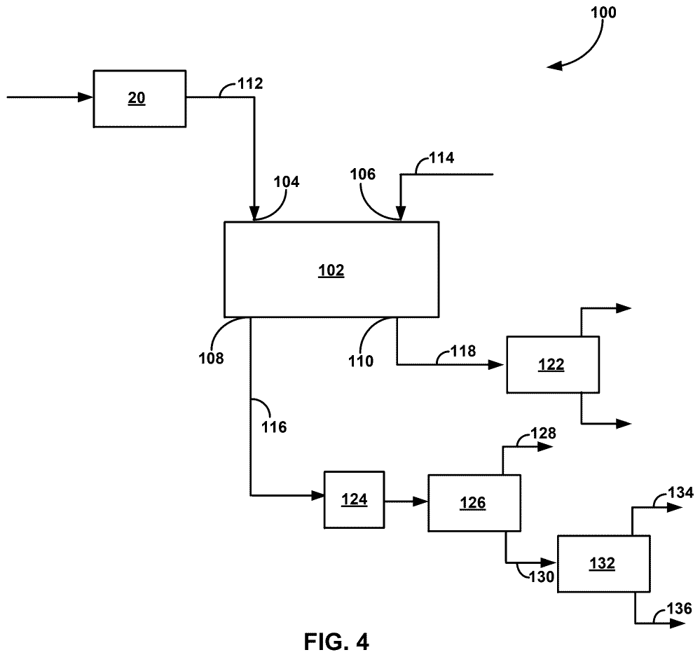

FIG. 4 is a block diagram illustrating one example extraction system 100 according to the disclosure in which a feedstock material, particularly a hemp seed material, is extracted. System 100 includes previously described flaking machine 20 and an extractor 102. Extractor 102 has a feed inlet 104 that can receive a flaked solid material. Extractor 102 also has a feed outlet 110 that can discharge the flaked solid particulate material after is has undergone extraction to remove extractable organic components (e.g., hemp oil) and has a lower concentration of extract than the incoming solid material. Extractor 102 also has a solvent inlet 106 configured to introduce a fresh solvent into the extractor and a solvent outlet 108 configured to discharge a miscella formed via extraction of extractable components from the solid material. In operation, the solid material being processed is contacted with solvent within extractor 102 (e.g., in counter current fashion), causing organic components soluble within the solvent to be extracted from the solid material into the solvent. Extractor 102 can process any desired solid material using any suitable extraction fluid.

In operation, a feedstock material is fed to flaking machine 20 to produce a flaked material 112. Flaked material 112 is then supplied to extractor 102 via feed inlet 104. A solvent 114 can enter extractor 102 via solvent inlet 106. The solvent 114 can contact the flaked solid material 112 in extractor 102 through one or more stages of extraction. Within the extractor, organic components (e.g., oil) soluble within solvent 114 can be extracted from the flaked solid material 112 in the solvent. This can produce a miscella 116 composed of solvent 114 and organic components (e.g., oil) extracted from the flaked solid material 112 into the solvent that discharges from solvent outlet 108. This can also produce a solvent-wet extracted material 118 that discharges from feed outlet 110.

The temperature conditions of solid material 112, solvent 114, and/or extractor 102 may be controlled to promote extraction of oil from the solid material into the solvent 114 within extractor 102. In specific implementations, the temperature of solid material 112, solvent 114, and/or extractor 102 may be controlled so that at least 80 wt % of the oil in solid material 112 is extracted and transferred to solvent 114 in extractor 102, such as at least 90 wt %, at least 95 wt %, or at least 98 wt %. The amount of oil extracted and removed in extractor 102 may be determined by comparing the oil content of incoming solid material 112 to the oil contact of extracted material 118.

The specific temperature to which solid material 112, solvent 114, and/or extractor 102 is controlled to ensure that the temperature is above that at which oil substantially extracts from the solid material into the solvent may vary depending on the type of feedstock being processed and the type of solvent used. In some examples, the temperature is greater than 50 degrees Celsius, such as greater than 60 degrees Celsius, or greater than 65 degrees Celsius. For example, the temperature of the miscella 116 generated by the extractor may range from 60 degrees Celsius to 90 degrees Celsius, such as from 65 degrees Celsius to 75 degrees Celsius, such as approximately 70 degrees Celsius.

The amount of time solid material 112 remains in contact with the solvent within the extractor (which may also be referred to as residence time) can vary, for example depending on the material being processed and the operating characteristics of the extractor. For a flaked hemp material, the material may be extracted with solvent for a period of time within a range from 30 minutes to 3 hours, such as greater than 30 minutes, greater than 45 minutes, or greater than 60 minutes. For example, the material may be extracted with solvent for a period of time within a range from 30 minutes to 120 minutes, such as from 45 minutes to 90 minutes. Providing sufficient residence time can help ensure that the oil is efficiently extracted from the hemp feedstock.

The relative amounts of incoming feed to feed inlet 104 and solvent to solvent inlet 106 can vary depending on the design parameters of the system and extraction objectives. In some example, extractor 102 is operated using a solvent ratio (weight of incoming solvent to inlet 106 divided by weight of the incoming solid material to feed inlet 104) greater than 1.0, such as a solvent ratio within a range from 1.5:1 to 5:1, such as from 2:1 to 4:1. Utilizing a solvent ratio where the amount of solvent supplied to the extractor is greater than the amount of solid material supplied to the extractor and be beneficial to efficiently extract high oil amounts from the feedstock.

A variety of different solvents may be used as solvent 114 in extractor 102. In different implementations, solvent 114 may be a polar protic solvent that is water soluble or a non-polar solvent that is water insoluble (e.g., hexane). In some examples, solvent 114 is a polar protic solvent that is water soluble. For example, solvent 114 may be an alcohol-based solvent. Example alcohol-based solvents that can be used include, but are not limited to, mono-hydroxyl or multi-hydroxyl (e.g., di-hydroxyl) alcohols having carbon chains 1 to 8 carbons in length, such as 1 to 4 carbons in length, or 2 to 3 carbons in length. For example, the alcohol-based solvent may be ethanol or isopropyl alcohol. In some examples, the alcohol-based solvent consists essentially of alcohol (e.g., with or without water). For example, the alcohol-based solvent may be a hydrous alcohol or an anhydrous alcohol solvent, such as hydrous or anhydrous ethanol. In some examples, the alcohol-based solvent has greater than 90 weight percent alcohol and less than 10 weight percent water, such as greater than 95 weight percent alcohol and less than 5 weight percent water, or greater than 98 weight percent alcohol and less than 5 weight percent water.

Miscella 116 and solvent-wet extracted material 118 produced by extractor 102 can be further processed following discharge from extractor 102. For example, to recover solvent from the solvent-wet extracted material 118 steam and further prepare the residual solids material for end use, the solvent-wet solids stream may be desolventized using mechanical and/or thermal desolventization devices. In the example of FIG. 4, system 100 includes a desolventizer 122. Desolventizer 122 can be implemented using one or more stages of mechanical and/or thermal treatment to remove solvent from the solvent-wet solids stream, thereby producing a dried extracted solid material (which may also be referred to as a desolventized extracted solid material). It should be appreciated that reference to a dried and/or desolventized solid material refers to a material that is comparatively dried and desolventized and does not require complete drying or desolventization or that the material be devoid of solvent. Rather, the material may be dried and desolventized to a practical level effective for downstream use and/or processing.

In some examples, desolventizer 122 mechanically presses the extracted solid material to remove residual entrained solvent from the extracted solid material. Additionally or alternatively, desolventizer 122 may heat the extracted solid material (the solvent-wet extracted material 118) produced by extractor 102 to vaporize solvent from the stream to produce a dried solid material. While desolventizer 122 may inject steam into the extracted solid material in some implementations, in other implementations, desolventizer 122 may desolventize the extracted solid material without adding moisture to the material during desolventizing. For example, desolventizer 122 may directly and/or indirectly heat the extracted solid material without injecting steam into the extracted solid material. Desolventizer 122 may indirectly heat the extracted solid material by passing a heat transfer fluid through a tray that the extracted material contacts while passing through a desolventizing vessel and/or through a jacket surrounding at least a portion of the desolventizing vessel. Additionally or alternatively, desolventizer 122 may introduce a heated gas substantially devoid of moisture (e.g., dried air, nitrogen) into an interior of the desolventizing vessel and extracted solid material therein. In some examples, when desolventizer provides thermal drying, the desolventizer operates at or below atmospheric pressure, e.g., to limit the temperature of the desolventizer.

Configuring desolventizer 122 to desolventize the extracted solid material without introducing additional moisture to the extracted solid material may be useful for subsequent solvent recovery. When using an alcohol solvent such as ethanol, the water and alcohol may form an azeotropic mixture that is challenging to separate for solvent recovery. Accordingly, desolventizing in the absence of added moisture may be useful in that the solvent vaporized by desolventizer 122 may have little or no water mixed with the alcohol that needs to be removed before the solvent can be recycled to extractor 102.

In different examples, desolventizer 122 can be implemented using a cooker, jacketed paddle mixer, bulk solids heat exchanger, and/or desolventizer-toaster. In any case, the solvent separated from the solvent-wet extracted solids stream via desolventizer 122 can be recycled back to extractor 102 for reuse (optionally with further processing, such as to decrease the water content in the solvent stream, before being returned to the extractor). In some examples, a stripping step is added before, after, or in conjunction with thermal drying, e.g., by introducing a non-solvent vapor such as steam or nitrogen to the material being dried.

Independent of whether extraction system 100 includes desolventizer 122, extractor 102 in the extractor system can produce miscella 116 that discharges through solvent outlet 108. Because the miscella contains solvent intermixed with extracted oil, the miscella stream may be further processed separate the solvent from the oil. The miscella 116 can be further processed to help separate the oil fraction of the miscella stream from the solvent fraction, thereby producing an extracted oil and recovered second solvent.

In the example of FIG. 4, system 100 includes a cooling unit 124 that is configured to receive miscella stream 116 and cool the stream to promote liquid-liquid phase separation between the aqueous alcohol-based second solvent component of the miscella and the extracted oil component of the miscella. Cooling unit 124 may be implemented using one or more heat exchangers or other thermal transfer devices that reduce a temperature of the miscella stream to a temperature effective to cause phase separation. In some examples, cooling unit 124 cools miscella 116 to a temperature less than 40 degrees Celsius, such as less than 30 degrees Celsius, or less than 25 degrees Celsius (e.g., a temperature ranging from 15 degrees Celsius to 25 degrees Celsius, such as approximately 20 degrees Celsius).

Cooling the miscella stream can produce a first solvent-rich phase and a first oil-rich layer. A compositional gradient may exist between the solvent-rich phase and the oil-rich phase formed by cooling the miscella stream. Depending on the relative amounts of solvent and water in the solvent-rich phase, the solvent-rich phase may be heavier than the oil-rich phase (allowing the solvent-rich phase to be separated off as a bottom phase and the oil-rich phase to be separated off as a top phase) or lighter than the oil-rich phase (allowing the solvent-rich phase to be separated off as a top phase and the oil-rich phase to be separated off as a bottom phase). For example, when using an alcohol-based solvent such as ethanol, a solvent-rich phase having ethanol and a comparatively high concentration of water may be heavier than the oil-rich phase, causing the oil-rich phase to be the light phase and the solvent-rich phase to be the heavy phase. However, when using a solvent-rich phase having ethanol and a comparatively low concentration of water, the oil-rich phase may be the heavy phase and the solvent-rich phase the light phase.

In either case, in the example of FIG. 4, extraction system 100 includes a separator 126 to separate the first solvent-rich phase from the first oil-rich phase. Separator 126 may be implemented using a decanter (e.g., gravity decanter) and/or other liquid separation device, such as a centrifuge and/or cyclone. Separator 126 can separate the solvent-rich layer from the oil-rich layer to produce a separated first oil-rich phase/stream 128 and a separated first solvent-rich phase/stream 130. As schematically illustrated, the separated first oil-rich phase/stream 128 is shown as being a light phase and the separated first solvent-rich phase/stream 130 as being a heavy phase, although the light and heavy phases may be reversed depending on the composition of the solvent phase. Regardless, the two separate streams may be recycled and/or further processed.

For example, the separated first solvent-rich stream 130 may be further processed with a secondary separator 132 to help remove residual oil from the stream. A secondary separation can be performed on the first separated solvent-rich stream to form a separated solvent stream and a second separated oil-rich stream. Secondary separator 132 may be, or include, one or more separation devices configured to further separate residual oil from the solvent in the separated first solvent-rich stream 130. In some implementations, secondary separator 132 includes a mechanical separation device, such as a centrifuge and/or a cyclone. Additionally or alternatively, secondary separator 132 may be configured to promote flocculation of the oil and/or solvent factions in separated first solvent-rich stream 130 to promote further separation of the two fractions.

For example, extraction system 100 may be configured so an amount of water is added to the separated first solvent-rich stream 130 to promote further phase separation between the oil component of the stream and the solvent component in the stream. The amount of water added to the first separated solvent-rich stream 130 may be comparatively small, such as an amount of water that is less than 10 weight % of a weight of the separated first separated solvent-rich stream 130, such as less than 5 weight %, less than 3 weight %, less than about 1 weight %, less than about 0.5 weight %, less than about 0.2 weight %, or less than about 0.1 weight %. In some examples, mixing equipment such as a static mixer, dynamic mixer, and/or homogenizer may be used to facilitate mass transfer between the phases and promote further phase separation.

Adding an amount of water to the separated first separated solvent-rich stream 130 can cause further liquid-liquid phase separation between the oil component in the stream and the solvent component (e.g., alcohol) in the stream. This can form a second solvent-rich layer phase separated from a second oil-rich phase. A compositional gradient may exist between the solvent-rich phase and the oil-rich phase formed by adding water to the first separated oil-rich stream.

In addition to or in lieu of adding water to promote further separation of the first separated solvent-rich stream 130, the secondary separator may further cool the first separated solvent-rich stream. For example, secondary separator 132 may cool the first separated solvent-rich stream 130 to a temperature less than a temperature to which the miscella is cooled by cooling unit 124. In these implementations, secondary separator 132 may include one or more heat exchangers or other thermal transfer devices that reduce a temperature of the first separated solvent-rich stream 130. In some examples, secondary separator 132 reduces the temperature of the first separated solvent-rich stream 130 to a temperature at least 5 degrees Celsius less than a temperature to which the miscella stream was cooled by cooling unit 124, such as a temperature at least 10 degrees Celsius less, at least 15 degrees Celsius less, at least 20 degrees Celsius less, or at least 25 degrees Celsius less. The cooling may promote phase separation between the aqueous solvent and the oil fractions.

In either case, secondary separator 132 may generate a second oil-rich phase 134 and a second solvent-rich phase 136. The separator (e.g., secondary separator 132) may be implemented using a decanter (e.g., gravity decanter) and/or other liquid separation device, such as a centrifuge and/or cyclone.

First solvent-rich phase 130 generated by separator 126 and/or second solvent-rich phase 136 generated by secondary separator 132 (when used) may be further processed and/or recycled. In some examples, first solvent-rich phase 130 generated by separator 126 and/or second solvent-rich phase 136 generated by secondary separator 132 are processed to remove water from the solvent-rich stream prior to further reusing the recovered solvent. For example, first solvent-rich phase 130 generated by separator 126 and/or second solvent-rich phase 136 generated by secondary separator 136 may be processed by a water removal unit to remove water, thereby generating a recovered second solvent (e.g., having the same composition as solvent 114).

When used, a water removal unit may be implemented using one or more stages of a molecular sieve (mole sieve), one or more stages of a pervaporation system, and/or one or more stages of a vapor permeation membrane. In general, a molecular sieve utilizes a material with small pores sized to allow comparatively small molecules (e.g., water) to enter the back for entrapment while comparatively larger molecules (e.g., alcohol) are unable to pass into the molecular pores. The molecular sieve can be periodically regenerated, e.g., by heating and purging with a carrier gas or under vacuum, to remove the separated water trapped in the sieve. By contrast, pervaporation generally involves a process of separating a mixtures of liquids by partial vaporization through a non-porous or porous membrane. The pervaporation process can proceed with initial permeation through a membrane by a permeate followed by evaporation into the vapor phase, generating a continuous stream of separated water from the residual solvent.

Extractor 102 can be implemented using any suitable type of extractor configuration. For example, extractor 102 may be an immersion extractor, a percolation extractor, or yet other type of extractor design. In one example, extractor 102 is an immersion extractor, which may be particularly effective for extracting hemp oil from hemp seed feedstock.

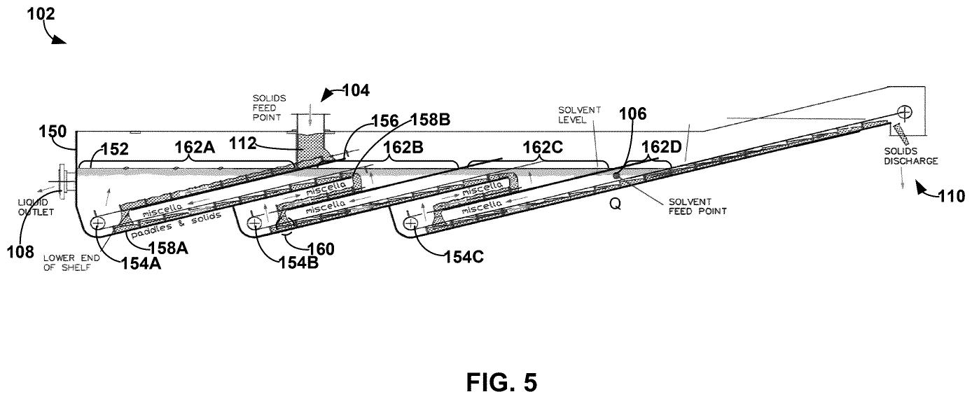

FIG. 5 is an illustration of an example immersion extractor configuration that can be used for extractor 102. As shown in this example, extractor 102 includes a housing 150 containing one or more extraction stages through which a material being processed travels in a countercurrent direction with an extraction solvent. Housing 150 includes a feed inlet 104 configured to receive a continuous or intermittent flow of flaked material 112. Extractor 102 also includes a feed outlet 110 configured to discharge the material 112 after some or all of the extract has been extracted into solvent flowing through the extractor.

To provide a flow of solvent passing through extractor 102, housing 150 also includes a solvent inlet 106 that receives solvent devoid of extract or having a comparatively low concentration of extract. A solvent outlet 108 is provided on a generally opposite end of housing 150 to discharge solvent having passed through extractor 102. As solvent travels through housing 150 from inlet 106 to outlet 108, the solvent flows in a countercurrent direction from the flow of material 112 passing through the extractor. The solvent intermixes with particulate material 112 within extractor 102, causing the extract carried by the solids material to transfer from the solids material to the solvent. Accordingly, in operation, solvent having a comparatively low concentration of extract enters at inlet 106 while solvent having an increased concentration of extract discharges at outlet 108. Likewise, fresh particulate material 112 carrying extract (e.g., oil) enters at inlet 104 while processed particulate material having a reduced concentration of extract is discharged at outlet 110. For example, in instances where flaked material 112 is flaked hemp hearts, solvent can extract hemp oil out of the material forming a miscella (the solution of oil in the extraction solvent) that is discharged through outlet 108.

Extractor 102 can be operated as an immersion extractor in which a pool or reservoir of solvent 152 is maintained in housing 150 to provide a desired solvent level inside the extractor. In such applications, incoming material 112 is immersed (e.g., submerged) in the pool of solvent 152 as it moves through extractor 150. In some examples, material 112 remains completely submerged in the pool of solvent 152 as it travels through extractor 102, e.g., except when adjacent inlet 104 and outlet 110. In other examples, material 112 travels above the pool of solvent 152 at different stages in extractor 102 before falling off the end of a conveyor and dropping back into the pool of solvent. As one example, extractor 102 may be implemented using a Model IV extractor commercially available from Crown Iron Works Company of Minneapolis, MN.

To contact particulate material 112 with solvent inside of extractor 102, the extractor has one or more conveyors that convey the material in a countercurrent direction through the pool of solvent 152. In the configuration of FIG. 5, for instance, extractor 102 has three conveyors 154A, 154B, 154C that convey particulate material 112 through the solvent pool 152 contained within housing 150. Particulate material 112 can travel along decks or trays 156 positioned inside of extractor 102 to define a bed of material. Each bed deck 156 may define a receiving end 158A and a discharge end 158B. In operation, particulate material 112 can drop onto the receiving end 158A of the bed deck 156 and then be conveyed along the bed deck by the conveyor until reaching the discharge end 158B. Upon reaching discharge end 158B, particulate material 12 can drop off or fall over the terminal edge of the bed deck, for example, onto a lower bed deck.

The vertical distance separating the discharge end 158B of an upper bed deck 156 from a receiving end 158A of a lower bed deck may provide a mixing or drop zone 160 through which particulate material 112 travels. For example, particulate material 112 dropping off the discharge end 158B of an upper bed deck 156 can mix and interact with solvent located between the upper bed deck and a lower bed deck in drop zone 160, e.g., as the solids material falls under the force of gravity toward the lower bed deck. A desired extract carried by the particulate material 112 can be extracted into the solvent within this drop zone as the solids material intermixes with the solvent within the drop zone. Increasing the number of bed decks 156 within extractor 102 and, correspondingly, the number of drop zones between bed decks, can increase the amount of extract recovered from a specific particulate material 112 being processed on the extractor.

Extractor 102 can have any suitable number of bed decks 156 arranged in any desired orientation. In the example, of FIG. 5, extractor 102 is illustrated as having six bed decks 156, although the extractor can have fewer bed decks or more bed decks. In addition, in this example, bed decks 156 are arranged at an inclined angle such that the bed decks are alternatingly sloped downwardly and upwardly. Bed decks 156 may be arranged in series with adjacent bed decks being vertically and/or laterally offset from one another to provide adjacent flow pathways over which particulate material 112 travels when passing through extractor 102. For example, bed decks 156 may be arranged in parallel to define a serpentine pathway along which particulate material 112 is conveyed through pool of solvent 152 between inlet 104 and outlet 110. In operation, particulate material 112 may travel along a downwardly sloped bed deck 156 before dropping onto an upwardly sloped lower bed deck, at which point the solids material reverses direction and travels laterally and vertically in an opposed direction from the direction of travel on the upper bed deck.

In the example of FIG. 5, particulate material 112 enters extractor 102 via inlet 104 and falls onto a first downwardly sloped bed deck. Conveyor 154A moves particulate material 112 from the receiving end of the first downwardly sloped bed deck to the discharge end of the first downwardly sloped bed deck, whereupon the solids material drops off of the deck through a first drop zone onto a first upwardly sloped bed deck. Conveyor 154A moves particulate material 112 from the receiving end of this first upwardly sloped bed deck to the discharge end of this bed deck, whereupon the solids material drops off of the deck through a second drop zone onto a second downwardly sloped bed deck. Conveyor 154B moves particulate material 112 from the receiving end of the second downwardly sloped bed deck to the discharge end of this bed deck, whereupon the solids material drops off of the deck through a third drop zone onto a second upwardly sloped bed deck. Conveyor 154B moves particulate material 112 from the receiving end of this second upwardly sloped bed deck to the discharge end of the bed deck, whereupon the solids material drops off of the deck through a third drop zone onto a third downwardly sloped bed deck. Conveyor 154C moves particulate material 112 from the receiving end of the third downwardly sloped bed deck to the discharge end of this bed deck, whereupon the solids material drops off of the deck through a fourth drop zone onto a third upwardly sloped bed deck. Finally, conveyor 154C moves particulate material 112 along this final bed deck out of the solvent pool 152 and discharges the processed solids material via outlet 110.

In some examples, the pool of solvent 152 contained within housing 150 is divided into fluidly interconnected sub-pools, e.g., to provide different equilibrium extraction stages. For example, bed decks 156 may provide physical barriers that separate each sub-pool from each adjacent sub-pool and prevent solvent from flowing through the bed deck. In such examples, solvent may flow around the discharge end 158B of each bed deck rather than through the bed deck, allowing the solvent to flow in a countercurrent direction from particulate material 112 through extractor 102. Other physical divider structures in addition to or in lieu of bed decks 156 can be used to separate the pool of solvent 152 in different sections.

In the example of FIG. 5, extractor 102 is illustrated as having four solvent pools 162A-162D. Each downwardly sloping bed deck 156 provides a barrier between adjacent pools with adjacent solvent pools being connected at the discharge end of a separating bed deck. In operation, each solvent pool of pools 162A-162D may have a different average extract-to-solvent concentration ratio to provide different stages of extraction. The concentration ratio may progressively increase from a lowest concentration adjacent solvent inlet 106 to a highest concentration adjacent miscella outlet 108.

Particulate material 112 processed in extractor 102 is conveyed out of solvent pool 152 and discharged through outlet 110 via a conveyor. In the configuration of FIG. 5, for instance, conveyor 154C conveys particulate material 112 out of solvent pool 152 towards discharge 110. Residual solvent retained by processed particulate material 112 can drain under the force of gravity back into solvent pool 152. For this reason, the final bed deck or discharge deck 156 along which particulate material 112 travels towards outlet 110 may be sloped upwardly away from solvent pool 152. Solvent carried with particulate material 112 out of solvent pool may drain down the sloped bed deck back into the solvent pool, helping to minimize the amount of solvent carried out extractor 102 by the processed solids material being discharged from the extractor.

After being processed on extractor 102, residual extracted solid matter 112 discharging from feed outlet 110 and miscella containing solvent intermixed with extracted oil can be further processed, e.g., as discussed above with respect to FIG. 3.

The following examples may provide additional details about hemp oil extraction systems and techniques in accordance with the disclosure.

### EXAMPLES

### Example 1: Flaking Characteristics

An experiment was conducted to evaluate the flaking characteristics of a hemp heart material. A pilot-scale flaker with dual counterrotating rolls was used as the test apparatus. Flakes were made at three roll gap sizes of 0.005″, 0.008″, and 0.012″ under otherwise consistent flaking conditions. A random sample of 25 flakes was then taken and size measured to evaluate the distribution and characteristics of the resulting flakes.

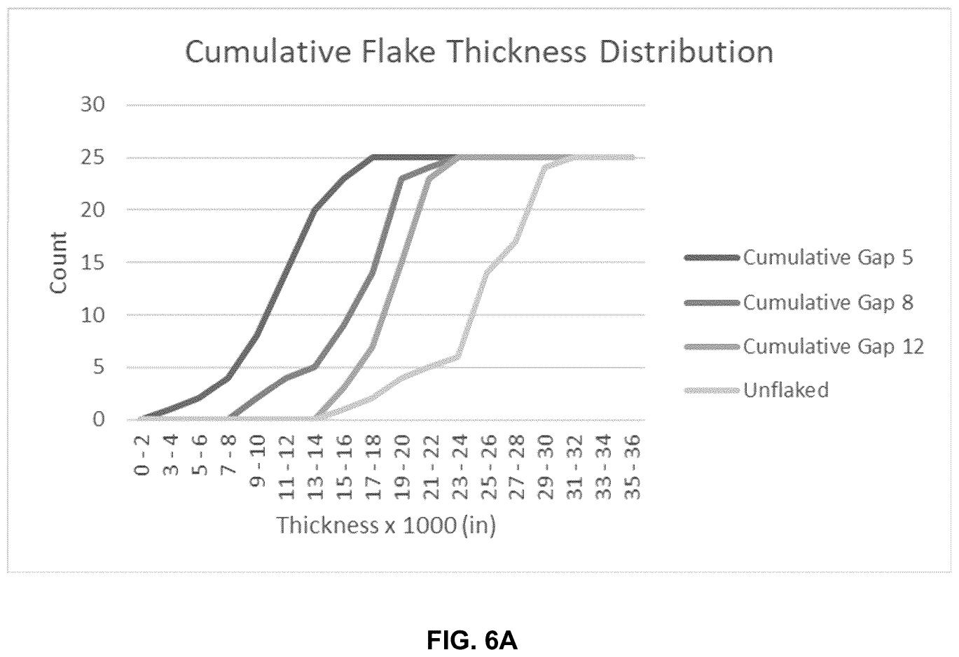

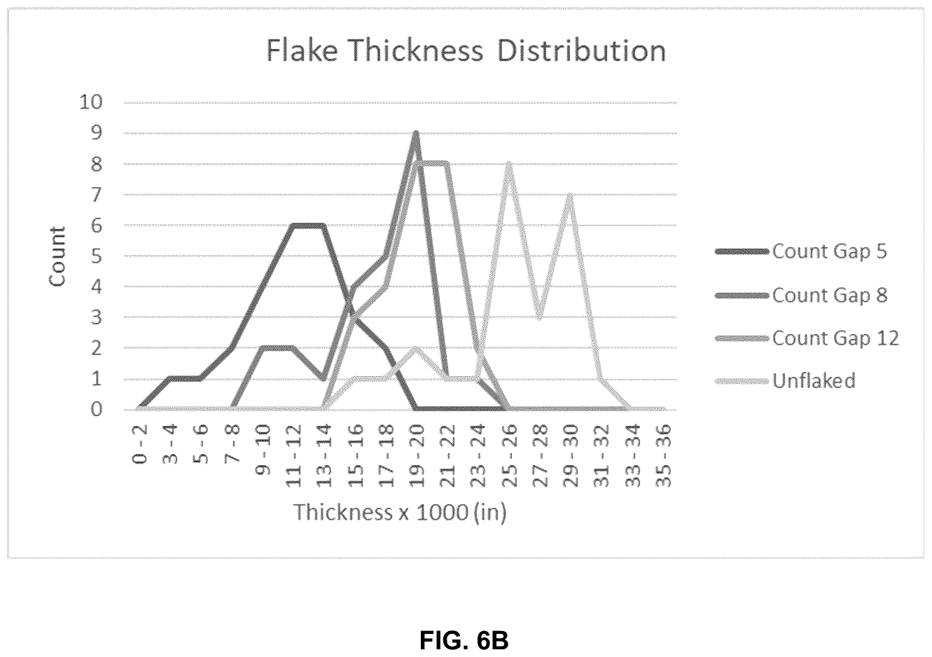

FIGS. 6A and 6B are plots of cumulative flake thickness distributions and flake thickness distributions for the experiments conducted. Table 1 below shows measured thickness data for each experimental run.

TABLE 1
Flake thickness data

| | | | | | |
|---|---|---|---|---|---|
| Gap | 1000 × in | 5 | 8 | 12 | Unflaked |
| Mean Thickness | 1000 × in | 11.7 | 17.2 | 19.8 | 25.6 |
| Std. Dev. | 1000 × in | 3.3 | 3.6 | 2.3 | 4.2 |

The data in Table 1 and FIGS. 6A and 6B show considerable differences in the distribution of flake thickness at each roll gap setting. Unexpectedly, the mean flake thickness observed was approximately 1.5 to 2.5 times the roll gap spacing set for the flaker. This indicated the hemp material exhibited considerable spring back and elastic re-expansion after flaking.

### Example 2: Extraction Kinetics

After forming a flaked hemp material at the three different roll gap settings discussed in Example 1 above, an experiment was conducted to quantify the extraction kinetics for an immersion style extraction for each of the flaked samples. The flaked hemp material was immersed in fresh ethanol solvent, with fresh solvent continuously feed to the extraction chamber and miscella collected fractionally.

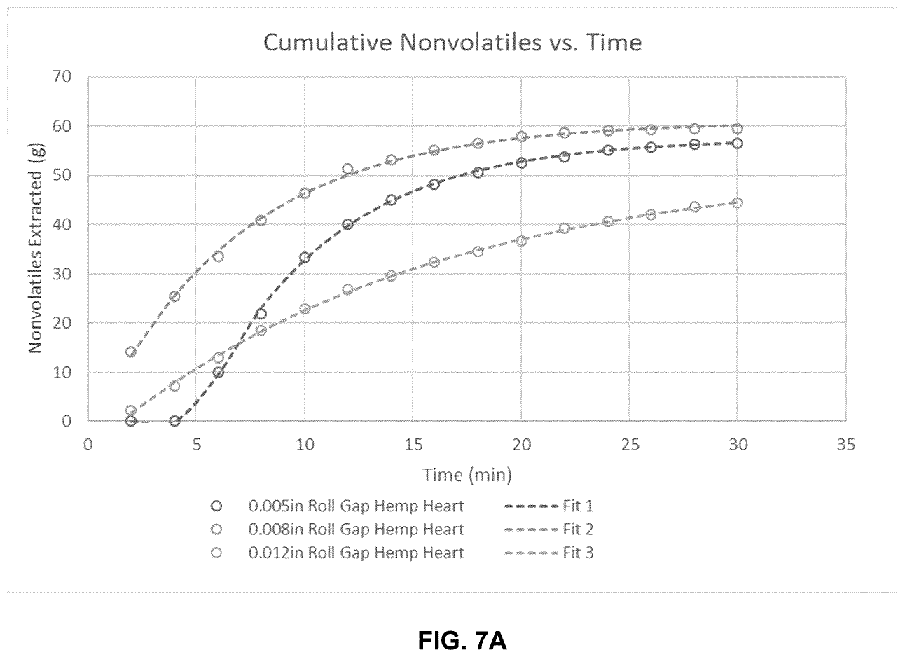

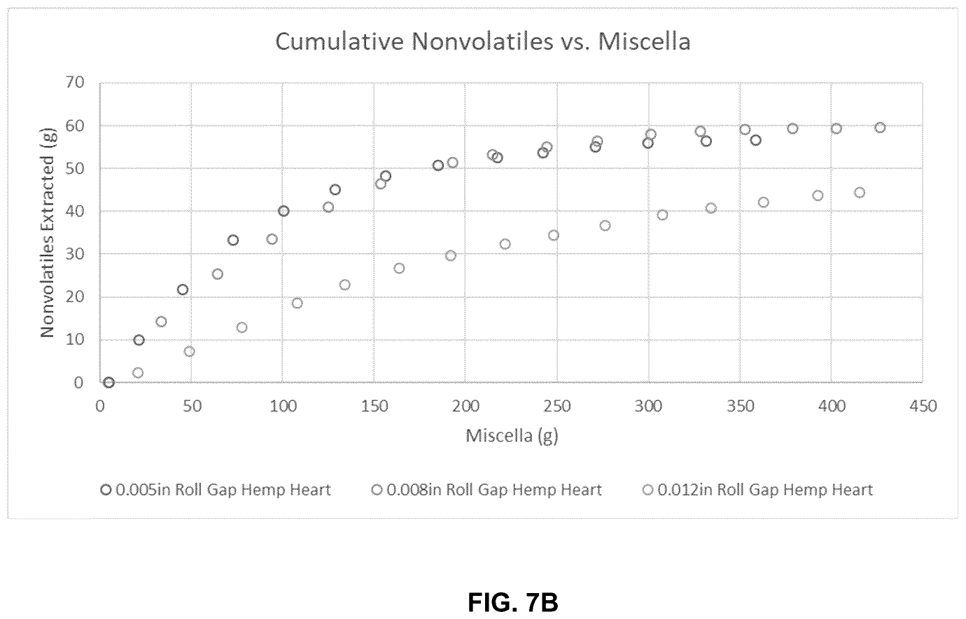

FIG. 7A is a plot of cumulative nonvolatiles extracted from the flaked hemp samples of different thickness versus time. FIG. 7B is a plot of cumulative nonvolatiles extracted from the flaked hemp samples of different thickness versus cumulative miscella recovered. The data show that the flaked hemp samples flaked at roll gap sizes of 0.005″ and 0.008″ exhibited similar extraction kinetics that were substantially faster than the extraction kinetics of the flaked hemp sample flaked at a roll gap size of 0.012″. This shows that faster extraction is achieved with thinner flakes, after accounting for variation in the initial delay due to drainage rate differences.

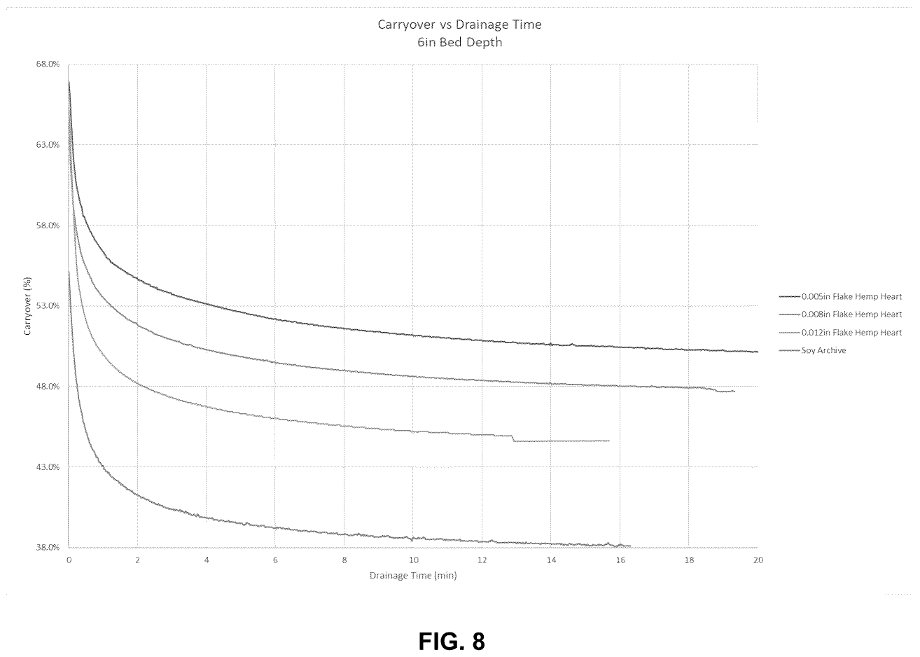

After extraction, the extracted flaked hemp material was withdrawn from the solvent in which it was immersed. A six inch bed of each extracted flaked hemp sample of different thickness was placed on a porous screen and solvent carryover and drainage times measured. The flaked hemp samples were compared to historical drainage data for soy flakes. FIG. 8 is a plot of solvent concentration in the extracted flaked hemp sample versus time for each of the hemp samples of different thickness. The data show that drainage time improves with increasing flake thickness and corresponding roll gap size.

1. A method comprising:
flaking a hemp material by passing the hemp material through a pair of rotating flaking rolls defining a nip clearance between the rotating flaking rolls within a range from 0.2 mm to 0.32 mm, thereby producing a flaked hemp material having a mean thickness within a range from 0.25 to 0.65 mm;
introducing the flaked hemp material into an extractor and contacting the flaked hemp material with a solvent, thereby producing an extracted hemp material and a miscella; and
separating the solvent from the miscella, thereby producing an extracted hemp oil.

2. The method of claim 1, wherein flaking the hemp material by passing the hemp material through the pair of rotating flaking rolls comprises driving the pair of rotating flaking rolls at a speed less than 2 m/s.

3. The method of claim 2, wherein each of the pair of rotating flaking rolls are driven at substantially a same speed.

4. The method of claim 1, wherein passing the hemp material through the pair of rotating flaking rolls comprises scraping a flaked hemp material from the pair of rotating flaking rolls using a scraper comprising a non-stick material.

5. The method of claim 1, wherein flaking the hemp material comprises flaking the hemp material at ambient conditions without preconditioning the hemp material.

6. The method of claim 1, wherein flaking the hemp material comprises cooling the hemp material below ambient temperature prior to flaking.

7. The method of claim 1, wherein:
introducing the flaked hemp material into the extractor and contacting the flaked hemp material with the solvent comprises introducing the solvent into a fresh solvent inlet of the extractor; and
a weight of the solvent supplied to the fresh solvent inlet of the extractor divided by a weight of the flaked hemp material supplied to the extractor defines a solvent ratio, and the solvent ratio is within a range from 2:1 to 4:1.

8. The method of claim 1, wherein the solvent is ethanol, and introducing the flaked hemp material into the extractor and contacting the flaked hemp material with the solvent, thereby producing the extracted material and the miscella, comprises extracting the flaked hemp material with the solvent at a temperature within a range from 65 degrees Celsius to 75 degrees Celsius.

9. The method of claim 1, wherein introducing the flaked hemp material into the extractor and contacting the flaked hemp material with the solvent, thereby producing the extracted material and the miscella, comprises extracting the flaked hemp material with the solvent for a period of time greater than 30 minutes, such as within a range from 45 minutes to 90 minutes.

10. The method of claim 1, further comprising, prior to introducing the flaked hemp material into an extractor, drying the flaked hemp material.

11. The method of claim 10, wherein drying the flaked hemp material comprises drying the flaked hemp material to a moisture content of 3 weight percent or less.

12. The method of claim 1, wherein the solvent comprises ethanol.

13. The method of claim 12, wherein the solvent comprises greater than 90 weight percent ethanol and less than 10 weight percent water.

14. The method of claim 1, wherein the hemp material comprises a hemp heart.

15. The method of claim 1, wherein the hemp material has an oil content within a range from 45 wt % to 55 wt %.

16. The method of claim 1, further comprising desolventizing the extracted hemp material.

17. The method of claim 16, wherein desolventizing the extracted hemp material comprises:
mechanically pressing the extracted hemp material to form a pressed extracted hemp material; and
thermally drying the pressed extracted hemp material.

18. The method of claim 17, wherein thermally drying the pressed extracted hemp material comprises thermally drying the pressed extracted hemp material at atmospheric pressure or vacuum pressure.

19. The method of claim 17, wherein thermally drying the pressed extracted hemp material comprises thermally drying the pressed extracted hemp material at temperature less than 110 degrees Celsius.

20. The method of claim 1, wherein separating the solvent from the miscella comprises:
cooling the miscella stream to form a solvent-rich phase and an oil-rich phase; and
separating the solvent-rich phase from the oil-rich phase.
